(19) United States
(12) Patent Application Publication
(10) Pub. No.: US 2022/0203001 A1
(43) Pub. Date: Jun. 30, 2022

(54) SMART CONNECTED BREAST PUMP

(71) Applicants: ResMed Corp., San Diego, CA (US); ResMed Pty Ltd, Bella Vista, NSW (AU)

(72) Inventors: Ryan Michael KIRKPATRICK, Sydney (AU); Sakeena DE SOUZA, Sydney (AU); Aniket SHARMA, Sydney (AU); Erika Lynn HAYDON, San Diego, CA (US); Lori Lynn BEHRENS, San Diego, CA (US)

(21) Appl. No.: 17/595,426

(22) PCT Filed: May 15, 2020
(86) PCT No.: PCT/US2020/033299

§ 371 Date: Nov. 16, 2021

Related U.S. Application Data
(60) Provisional application No. 62/849,516, filed on May 17, 2019.

(51) Int. Cl. A61M 1/06

(52) U.S. Cl. CPC A61M 1/0693; A61M 2205/3344; A61M 2205/52; A61M 2205/3592; A61M 2205/3584

(57) ABSTRACT

A system and method for increasing the efficiency of a breast pump device is disclosed. The system includes a breast pump device with a vacuum pump to provide suction force to a flange attached to the breast of a user. The device includes a controller that controls the pump with different modes of pumping operation including a prime mode and a pump mode. The pump device may include a pressure sensor that provides data to a transceiver sending data to an external device. The flange unit may include a substantially circular rim with a layer configured to contact the skin of a user to convey the suction force. The system may include milk containers that include a machine scannable external identification tag.

### PRIORITY CLAIM

The present disclosure claims priority from U.S. Provisional Application Ser. No. 62/849,516, filed on May 17, 2019. The contents of that application are hereby incorporated by reference.

### TECHNICAL FIELD

The present disclosure relates generally to a breast pump, and more specifically to different mechanisms and systems for efficient and comfortable use of a breast pump.

### BACKGROUND

Almost all mothers who deliver at term have the capacity to provide milk for their babies. Once lactation begins, the baby regulates milk production by local inhibition of milk synthesis independently in each breast as milk accumulates between breast feedings. However, lactating women need to regularly express milk to maintain a good milk supply. Without expressing milk regularly, the milk supply is liable to drop. However, there may be situations where mothers have issues with lactation. For example, mothers who deliver premature children may have a low milk supply. In addition, some preterm babies cannot feed directly from their mother due to either sickness or the immature co-ordination of the suck, swallow and breath reflex. This results in mothers having to supplement their milk production, and thereby provide sufficient breast milk for their babies. Further, for mothers with other commitments such as a job, that prevent being near the baby during lactation periods, stored milk from the breast pump can be used for feeding. Thus, working mothers, who are not at home for long periods of time, need to pump breast milk in order to have it available for their babies. Having stored milk from breast pumps is also useful for other situations where the mother is away from home for an extended time period and cannot produce milk for the baby on demand.

Thus, these mothers often use a breast pump for both the initiation and maintenance of their milk supply. Breast pump systems typically have a pump that creates suction through a hose that is attached to a funnel shaped flange. A vacuum unit exerts a suction force to the breast thus sucking milk from the breast by applying a vacuum to the nipple. This process is analogous to the sucking action of a baby during breast feeding. The flange is placed over the breast and the suction draws milk from lactation. The flange is typically connected to a housing that has a coupling where a container such as a bag or a bottle may be used to collect the milk. When milk is expressed from the breast the milk flows through the flange via the coupling to a container such as a bottle or a bag.

Known breast pumps include manual breast pumps that rely on the mother to manually operate the pump to create the vacuum force. Such manual pumps may be difficult to use and may be unwieldy. Other known breast pumps use electrical power to power the pump to create the vacuum force. Some electrically powered breast pumps require an AC power source to operate, thus making them rely on being near a power outlet. Certain electrical pumps are battery driven to allow use anywhere. However, battery powered pumps may be inefficient because the pump motor is continuously running to maintain vacuum force during the milk extraction process thus draining the battery. Existing pumps have a standard flange that may be uncomfortable for some users based on the skin contact or fit on the body. Current flanges rely on plastic or silicone interfaces with basic geometry that may not be optimal for comfort of the user since there is often only one size.

Further, most existing breast pump devices do not have any method to track usage or operation. Thus, power may be wasted on inefficient or unnecessary pumping as pressure and requirements may differ for different users.

Thus there is a need for a miniaturized breast pump that efficiently uses power. There is a further need for a breast pump that collects data to provide individualized settings for the pump. There is a further need for increased comfort and efficiency in the flange to improve the breast pumping experience. There is also a need for a breast pump system that allows tracking of milk production to assist in management of the milk.

### SUMMARY

One disclosed example is a superior at home solution than existing breast pump devices through providing a combination of improved user experience, and clinical understanding of individual needs. The disclosed example system includes a pump device, a pressure sensor, a breast interface, a milk container, and an application for a mobile device in communication with the pump device. The pump device is a miniature and wearable breast pump device. The device may include Bluetooth® or other form of wireless communication with a mobile device. The pump device has little to no user interface and is controlled via the application (“app”) run on the mobile device. An “Autoset” mode of the pump device will first learn the user preference after an initial manual set up by the user, then provide a user customized mode upon subsequent use. For example, the user may manually set the device to an initial prime mode, which massages the breast to simulate breast feeding. In the prime mode, the negative pressure in the breast interface surrounding the breast fluctuates. This prime mode is intended to initiate milk production via the “let-down” reflex. Once milk starts to produce, the user may switch to a pump mode, which is a more constant negative pressure that continues to extract milk from the breast. The autoset mode, via the application and pump device, will learn the user's inputs e.g., duration of the ‘prime mode’, when the pump mode was activated, and how much milk is produced.

The system may also include a pressure sensor proximal to the breast interface to measure pressure changes in the interface and send the signal to the smartphone via the pump device. The pressure sensor can be used to estimate milk production by e.g. machine learning, under a given pressure waveform and duration. The pressure sensor can also be used to monitor the pressure required to draw milk. The pressure sensor can also be used to determine any air leak in the system. Leak detection can be used to indicate to the user that a leak may be occurring via the breast interface. Leaks may be determined by a sudden decrease in negative pressure at the interface under a given negative pressure generated by the device.

The system also includes an improved breast interface such as a shield or a flange that contacts the skin of a user. The breast interface has enhanced seal performance on the breast of the user. Seal and comfort may be improved by the selection of tactile surface finishes and/or material selection that improve compliance and tactile comfort. For example, silicone interfaces may be flocked or have particular surface textures that improve the surface feel or reduce friction, thereby improving comfort. In addition, surface features may be added to improve seal performance such as by providing adhesive sections to better engage the breast. Seal and comfort may also be improved by material selection. For example, foam interfaces are known to improve seal and comfort of facial masks due to their improved compliance in comparison to conventional facial masks. Foam may comprise an increased spring rate over materials such as silicone or plastic flange seals. Foam may also improve seal and comfort by even distributing load on the breast during use. Alternatively, textile technology has improved over time, enabling complete 3D structures to be produced. Thus, textile seals with complex geometries can be constructed to improve seal and comfort of breast interfaces. Textiles are known to be more stretchable than silicone, while being able to maintain significantly thin cross sections in use.

The system may include a breast milk container such as a bag that may enable tracking of stored milk. The milk stored in the bag may be tracked by unique QR codes or other identifiers that may be scanned by the smartphone and tracked via a database in the app. The user may be informed of data such as when the bag was used, how much milk was produced and when it is expected to expire. The application may also provide a systematic scheduling of when to use a specific bag. The bags may include a visual temperature indicator, which indicates when the milk is too hot to drink.

The application may control the pump device via preprogrammed algorithms to improve the pumping experience. The pump may be controlled to generate a desirable negative pressure in the interface to enhance pumping efficacy resulting in higher milk production and also avoiding discomfort. The settings may be auto selected via tracking of previous inputs into the device/app by the user, e.g. via the autoset function explained above. In addition, the pump device may track efficacy via the pressure sensor and learn optimal settings for future use and adapt the settings accordingly. All usage information may be tracked and logged e.g. how often the device was used, when, where and how much milk was produced. This information may be provided back to the user to track progress and also determine optimal usage times and settings. The information may also be provided to clinicians/lactation consultants, so they may tailor advice. The usage data could also be provided to insurers to track device usage. Data may also be collected to provide commercial insights.

The application may provide a portal to have video links to lactation consultants and doctors. The application may also provide positive reinforcement of usage e.g. rewards for using the pump. The application may provide the user with pre-set or customized scheduling of pumping such as through calendar reminders and assistance to prevent mastitis or other breast engorgement complications. The application may also provide the user with education on pumping or access to social groups. The application may have a portal to online resupply of consumables. The application may track usage and provide reminders or an automated ordering function to replace consumable as required.

In accordance with one aspect of the present technology, there is disclosed a breast pump device including a vacuum pump to provide suction force to a flange. The flange is configured to attach to a chest area of a user around a breast of the user. The vacuum pump has different settings to control the suction force during a pumping operation. A controller is configured to control the pump. The controller is operable to provide different modes of pumping operation. The modes include an initial prime mode to provide fluctuating negative pressure within the flange to simulate feeding, and a pump mode providing a more constant negative pressure.

A further implementation of the example breast pump device is an embodiment including a transceiver in communication with an external device. The transceiver is configured to transmit data associated with the pumping operation; and receive control commands for the controller Another implementation is where the external device is operative to learn optimal settings for the user based on transmitted data specific to the user. Another implementation is where the transmitted data include at least one of the duration of the prime mode, how much milk is produced, and the pressure within the flange during the pumping operation. Another implementation is where the optimal settings are provided in the control commands by the external device. Another implementation is where the optimal setting is a time for pumping operation. Another implementation is where the external device is a mobile device associated with the user. Another implementation is where the mobile device is in communication with an application server. Another implementation is where the external device is a networked server. Another implementation is where the device includes a pressure sensor coupled to the flange. The pressure sensor is configured to provide pressure data associated with the pumping operation.

In accordance with another aspect of the present technology, there is disclosed a breast pump device including a vacuum pump to provide suction force to a flange. The flange is configured to attach to a chest area of a user around a breast of the user. The vacuum pump has different settings to control pressure of the suction force. A pressure sensor is coupled to the flange. The pressure sensor is configured to generate pressure data representative of air pressure in the flange. A controller is configured to control the pump. A transceiver is in communication with an external device. The transceiver is configured to transmit pressure data received by the controller from the pressure sensor.

A further implementation of the example breast pump device is an embodiment where the pressure sensor is a gauge type transducer. Another implementation is where the controller is further configured to determine the duration of a pumping session, and send the duration data to the transceiver. Another implementation is where the external device is operable to determine an estimate of milk production based on the received pressure and duration data. Another implementation is where the external device is operable to determine the pressure required to be applied by the pump to draw milk. Another implementation is where the pressure data is used to determine an air leak based on a decrease in sensed negative pressure.

In accordance with another aspect of the present technology, there is disclosed a flange unit for attachment to a breast pump. The flange unit includes a connector to a hose providing suction force from the breast pump. The flange unit also includes a flange having a coupling to the connector and an opposite substantially circular rim. The substantially circular rim includes a layer configured to contact the skin of a user to convey the suction force to the skin.

A further implementation of the example flange unit is where the layer has a microtexture to facilitate comfort and sealing contact with the skin. Another implementation is where the layer comprises a foam material to distribute the load of the flange on the skin. Another implementation is where the layer includes a complex geometry to improve the seal of the flange with the skin. Another implementation is where the shape of the flange is custom sized for the user based on measurements of physical features of the user.

In accordance with another aspect of the present technology, there is disclosed a support system for operation of a breast pump device. The breast pump device includes a vacuum pump to provide suction force to a flange. The flange is configured to attach to chest area of a user around a breast of the user. A controller is configured to control the pump according to different modes of pumping operation. The modes include an initial prime mode to provide fluctuating negative pressure within the flange to simulate feeding, and a pump mode providing a more constant negative pressure. The device also includes a transceiver. A network interface receives data associated with the pumping operation from the transceiver. A database is coupled to the network interface to store the data received from the transceiver. The database includes data associated with the user. A processor is operable to collect the data received from the transceiver, process the data, and provide the processed data to the database.

A further implementation of the example support system is where the breast pump device is one of a plurality of breast pump devices supported by the support system. Another implementation is where the processor is operable to determine initial settings for the pump device based on analysis of data from the plurality of breast pump devices. Another implementation is where the processor is operable to determine optimal scheduling for pumping based on analysis of data from the breast pump devices. Another implementation is where the processor is operable to determine a time for replacement of the vacuum pump or the flange of the pump device. Another implementation is where the processor is operable to determine the time for replacement based on analysis of data from users of the breast pump devices with similar characteristics to the user. Another implementation is where the processor is operable to communicate to a computing device associated with the user a selected assistance medium. The assistance medium is selected based on analysis of the received data.

In accordance with another aspect of the present technology, there is disclosed a milk container operable for connection to a breast pump. The milk container includes a coupling operable to be connected to a breast pump device. The milk container also includes an external identification tag encoding identification information associated with the container. The external identification tag is machine-scannable. The container includes a containment compartment for storing milk.

A further implementation of the example milk container is where the containment compartment is transparent. Another implementation is where the compartment includes a volume scale configured to show the amount of milk stored in the container. Another implementation is where the container includes a visual temperature indicator on the containment compartment configured to show the temperature of the stored milk. Another implementation is where the container is a bag. Another implementation is where the container is a bottle. Another implementation is where the identification tag is a bar code. Another implementation is where the identification tag is a QR code. Another implementation is where the identification tag is printed on the container. Another implementation is where the container includes a human-readable identification number associated with the identification information.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a block diagram of an example support system that facilitates data collection and analysis for efficient usage of a breast pump system by a user;

FIG. 2A is a block diagram of the components of an example breast pump device for use in the breast pump system in FIG. 1;

FIG. 2B is a block diagram of the pressure sensor module in the example breast pump system in FIG. 2A;

FIG. 3 is a perspective view of an example breast pump system in FIG. 1;

FIG. 4A is a perspective view of the flange unit of the breast pump system in FIG. 3;

FIG. 4B is a close up perspective view of the flange of the flange unit for the breast pump system in FIG. 4A;

FIG. 4C is a view of a flange unit attached to a user for operation of the breast pump system in FIG. 3;

FIGS. 5A-5H are images for different examples of textured surfaces for the contact surfaces of the flange of FIG. 4B;

FIG. 6 is a flow diagram of a process for scanning a user and using the scanned data to select an appropriate flange for a breast pump;

FIG. 7 is a flow diagram of the process of adjusting settings on the breast pump system in FIG. 1;

FIG. 8A is an image of a user interface on a mobile device associated with a user of the breast pump system in FIG. 1;

FIG. 8B is another image of the user interface on the mobile device showing production information from the breast pump system in FIG. 1; and

FIG. 9 is a perspective view of a milk container that may be tracked for milk production from the breast pump system in FIG. 1.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

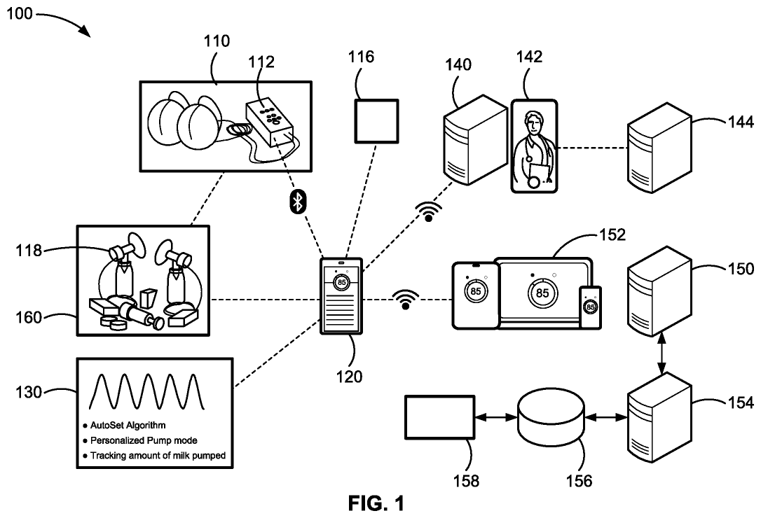

FIG. 1 shows an example support system 100 for the operation of a breast pump system 110. The breast pump system 110 includes a pump device 112, and a breast interface known as a flange unit 114. One or more physiological sensors 116 may be in contact with a user of the breast pump system 110. As will be explained below, the breast pump system 110 includes a transceiver that communicates with a computing device such as a mobile device 120 that is associated with the user. The mobile device 120 and physiological sensors 116 are co-located with a user and the breast pump system 110.

The mobile device 120 includes an application 130 that receives data produced from the pump system 110. As will be explained, the data may include data on the volume of milk pumped, pressure applied by the pump, operational settings of the pump device 112 and the like. In this example, the application 130 generates a user interface on the mobile device 120 to display received data to the user. The application 130 may include an algorithm that can determine settings of the pump device 112 as will be explained below. The application 130 may determine a personalized pump mode for the user. The application 130 may also track the amount of milk pumped during each operation of the breast pump system 110. The application 130 may have other functions as will be explained below. The functions of the application 130 and the mobile device 120 may alternatively, or additionally, be provided by the pump system 110 if appropriate hardware is provided.

The system 100 includes a health care provider (HCP) server 140 that may be in network communication with the mobile device 120. The health care provider server 140 may allow contact with health care providers such as lactation consultants, doctors, or OSCAR health through corresponding communication devices such as another mobile device 142. The health care provider server 140 may receive the data from the application 130 to provide better support for the user in relation to operation of the pump system 110. The health care provider server 140 may access an electronic medical records (EMR) server 144.

The system 100 also includes a clinical server 150 and a payor server 152. The servers 150 and 152 may receive the data from the application 130. The payor server 152 analyzes user data to support payments to the user in relation to operation of the pump system 110. The clinical server 150 may be provided with an application/data analysis server 154 that compiles data from multiple users in a database 156. For example, the data server 154 may execute machine learning applications 158 that may analyze the data from the pump system 110 and provide initial settings, predictions for milk production and other optimization of components in the system 100.

The system 100 also includes a supplier system 160 that may be provided data from the application 130 for replacement of consumables 118 for the pump system 110 such as the flange, milk containers, pump parts, etc. The supplier system 160 includes infrastructure such as servers to activate a supply chain to deliver the replacement consumables to the user or a technician to service the pump system 110. As will be explained below, the supplier system 160 may access user data to predict the replacement of consumables for the pump system 110.

In the system 100, the servers and devices are all connected to, and configured to communicate with each other over, a wide area network, such as the Internet. The connections to the wide area network may be wired or wireless. The user may be associated with a computing device such as the mobile device 120. The computing device may also be a personal computer, mobile phone, tablet computer, or other device. In this example, mobile device 120 is configured to intermediate between the user and the remotely located entities of the system 100 over the wide area network. In this example, this intermediation is accomplished by the software application 130 that runs on the mobile device 120. In one example, the application 130 may be a dedicated application. In another example, the application 130 may be a web browser based application that interacts with a web site hosted by an application server supervised by an entity such as a health care provider.

In an alternative implementation of the system, the sensors 116 and the breast pump system 110 communicate with the mobile device 120 via a local wired or wireless network (not shown) based on a protocol such as Bluetooth®. It is to be understood that the system 100 may support multiple breast pump systems that are similar to pump system 110 for multiple users. Such breast pump systems with respective users may also have respective associated computing devices similar to the mobile device 120 and associated HCP servers such as the HCP server 140 (possibly shared with other user).

As explained above, the breast pump system 110 is configured to transmit the data to servers such as the HCP server 140 or the clinical server 150 to provide different support services to the user. The servers may receive the data from the breast pump system 110 according to a “pull” model whereby the breast pump system 110 transmits the data in response to a query from the server. Alternatively, the server may receive the data according to a “push” model whereby the breast pump system 110 transmits the data to the server as soon as it is available after a session of operating the breast pump system 110.

Data received from the breast pump system 110 may be stored and indexed by the data server 154 so as to be uniquely associated with the breast pump system 110 and therefore distinguishable from data from any other breast pump system(s) participating in the system 100. In this regard, although only one breast pump system 110 is illustrated in FIG. 1 for ease of explanation, as mentioned above, the system 100 may have multiple breast pump systems.

In this example, the data server 154 may be configured to calculate summary data for each pumping session from the data received from the breast pump system 110. Summary data variables for a session comprise summary statistics derived by pump data or pressure data.

In an alternative implementation, the breast pump system 110 calculates the summary variables from the pump data stored in internal memory at the end of each pumping session. The breast pump system 110 then transmits the summary variables to the data server 154 according to the “push” or “pull” model described above. Alternatively, a memory of the breast pump system 110 that may store pump session data is in removable form, such as an SD memory card. The removable memory device is removed from the breast pump system 110 and inserted into a card reader in communication with the server 154. The pump session data is then copied from the removable memory to the memory of the server 154.

In still a further alternative implementation, the breast pump system 110 may be configured to transmit the data to the mobile device 120 via a wireless protocol such as Bluetooth®, which receives the data as part of the application 130. The mobile device 120 then transmits the data to the server 154, possibly along with summary data. The server 154 may receive the data from the mobile device 120 according to a “pull” model whereby the mobile device 120 transmits the data in response to a query from the server 154. Alternatively, the server 154 may receive the data according to a “push” model whereby the mobile device 120 transmits the data to the server 154 as soon as it is available after a pumping session.

The HCP server 140 is associated with the health/home care provider (which may be an individual health care professional or an organization) that is responsible for the natal care of the user. An HCP may also be referred to as a DME or HME (domestic/home medical equipment provider). The HCP server 140 hosts different processes for HCP specific purposes. One function of the HCP server process is to transmit data relating to the user to requesting data servers such as the data server 154, possibly in response to a query received from a data server.

The EMR server 144 contains electronic medical records (EMRs), both specific to the user and generic to a larger population of breast pump users with similar characteristics to the user. An EMR, sometimes referred to as an electronic health record (EHR), typically contains a medical history of the user including previous conditions, treatments, co-morbidities, and current status. The EMR server 144 may be located, for example, at a hospital where the user has previously received treatment. The EMR server 144 may be configured to transmit EMR data to a data server, possibly in response to a query received from a data server.

The HCP server 140, the EMR server 144, clinical server 150, payor server 152, and the data server 154 may all be implemented on distinct computing devices at separate locations, or any sub-combination of two or more of those entities may be co-implemented on the same computing device.

The data server 154 may also be configured to receive data from a user computing device such as the mobile device 120. Such may include data entered by the user to the application 130 or behavioural data about how the user is interacting with the application 130. The behavioural data may also include data indicating how the user is interacting with the breast pump system 110.

The data server 154 may also be configured to receive physiological data from the one or more physiological sensors 116. The sensors 116 may include Doppler radar motion sensors, accelerometers, thermometers, scales, or photoplethysmographs, each of which is configured to provide physiological data (biomotion, physical activity, temperature, weight, and oxygen saturation respectively) of the user.

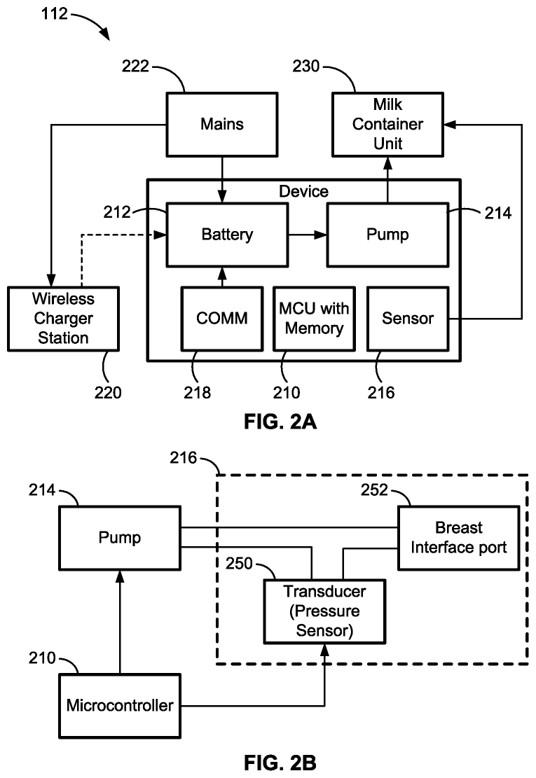

FIG. 2A is a block diagram of the electronic components of the pump device 112 in the pump system 110 in FIG. 1. In this example, the pump device 112 is miniaturized and may be worn by a user for pump operation. The pump device 112 includes a main controller unit 210 that is coupled to a battery 212, a vacuum pump 214, a pressure sensor unit 216, and a communication module 218. The battery 212 may be charged by a wireless charger 220 or a main power unit 222. The vacuum pump 214 is connected via a hose to a milk container 230. The milk container 230 is also connected to the pressure sensor unit 216. In this example, the main controller unit 210 may be a microprocessor that supervises the operation of the pump device 112, and gathers and analyzes data from the pump device 112.

In this example, the communication module 218 may include a short-range wireless communication protocol such as Bluetooth® to be paired with a mobile device such as the mobile device 120 in FIG. 1. Alternatively, or additionally, the communication module 218 may include a wireless protocol transceiver that allows direct communication with a network to send data to external servers, such as the servers 150 and 152 in FIG. 1.

FIG. 2B is a block diagram of the pressure sensor unit 216. The pressure sensor unit 216 includes a pressure sensor 250 and a breast interface port 252. In this example, the pressure sensor is a gauge type transducer from the Amphenol NPA series 5 psi sensor. Of course, other types of pressure sensors, such as non-gauge sensors, may be used. In this example, the pressure sensor 250 measures differential pressure between the suction (negative pressure relative to atmospheric pressure) created by the vacuum pump 214 and external air pressure. One port of the sensor 250 is connected to the breast interface port 252 which will be under suction from the vacuum pump 214. The other port of the sensor 250 is connected to measure external atmospheric pressure. In this manner, the output of the pressure sensor 250 is the difference between the negative pressure and external air pressure.

Thus, the pressure data from the pressure sensor 250 may be used to measure pressure changes in the flange unit 114 in FIG. 1 during the pumping operation which changes the negative pressure created by the suction force from the vacuum pump 214. The system 100 may also allow sending the data signals from the pressure sensor 250 to an external device such as the mobile device 120. Data from the pressure sensor 250 can be used to estimate milk production from the pump system 110 by determining a pressure waveform and duration of operation of the vacuum pump 214 during milking.

Data from the pressure sensor 250 can also be used to determine any air leak in the pump system 110. Leak detection can be used to indicate to the user that a leak may be occurring from the interface between the flange unit 114 and the breast of the user. Such leaks waste energy as the entire suction force from the vacuum pump 214 cannot be applied to the flange unit 114, which impairs the pumping operation. A leak may be determined by a sudden decrease in negative pressure at the interface with the breast under a given negative pressure generated by the pump system 110. An alert may be provided by the vacuum pump 214 or the mobile device 120 to the user via an audio or visual alarm. Alternatively, a leak may be reported via message or icon generated on the display of the mobile device 120 by the application 130. After compiling the pressure data from multiple pumping sessions, an application server may determine pressure curves associated with a particular user based on machine learning.

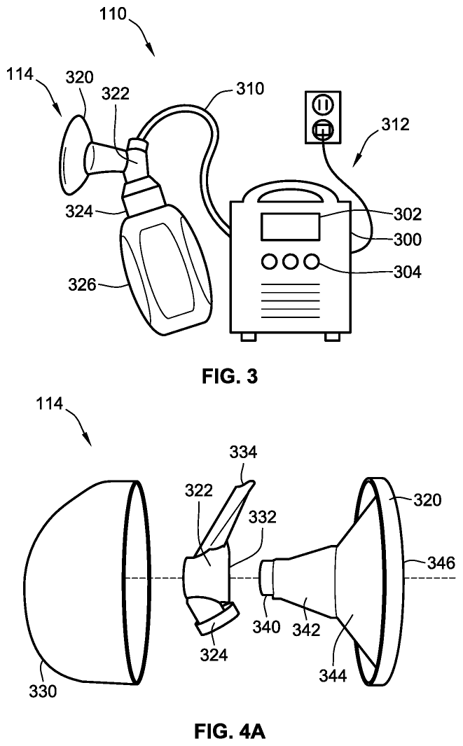

FIG. 3 is a perspective view of the components of the pump system 110, the pump device 112, and the flange unit 114 in FIG. 1. The pump device 112 includes a housing 300 that includes a control display 302 and a control panel 304. The control panel 304 allows a user to make adjustments to settings, such as pressure, or cycling intervals, for a vacuum pump such as the vacuum pump 214 in FIG. 2A. In this example, the control display 302 may include readings relating to the operation of the pump device 112. The suction generated by the vacuum pump 214 is created in a hose 310 that attaches the pump device 112 to the flange unit 114.

Alternatively, some or all of the functions of the control panel 304 and the display 302 may be generated by the application 130 on the display of the mobile device 120. It is preferable for the pump device 112 to have little to no user interface and be controlled by the mobile device 120. Thus, it is preferred that the pump device 112 has only a simple on and off switch, such that it may be used without a mobile device under a simple mode of use. The below described functions may be controlled by more complex settings that can be utilized via an application interface generated on the display of the mobile device 120 by the corresponding application 130. The mobile device 120 may be enabled to set customized pumping modes for the pump device 112.

The flange unit 114 includes a flange 320 that is configured to be attached to the chest area of a user around the breast to be pumped. The flange 320 is coupled to a plastic valve housing 322 that includes a coupling 324 that holds a removable milk container such as a bottle 326. Alternatively, the milk container may be a bag, or other suitable container. The milk obtained by the pump action is stored in the milk container for later use.

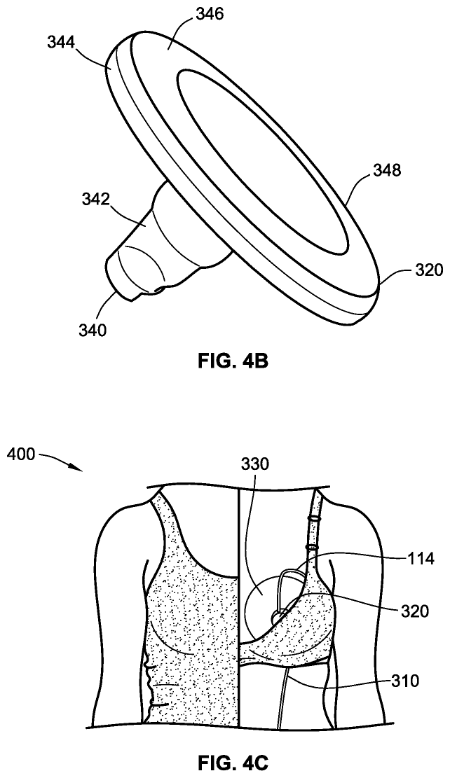

FIG. 4A is a perspective view of the components of the flange unit 114 in the pump system 110 in FIG. 1. FIG. 4B is a close-up perspective view of the flange 320. The flange unit 114 includes a hemispheric plastic cover 330 that protects the valve housing 322 and the flange 320 when the pump system is in operation. FIG. 4C is a view of the flange unit 114 including the flange 320 and cover 330 in FIG. 4A-4B placed on a user 400 for purposes of the pumping operation.

The hemispheric cover 330 is placed over the breast as shown in FIG. 4C. The flange 320 is placed over the nipple of the breast and conveys suction force generated by the vacuum pump 214. As shown in FIG. 4A, the plastic valve housing 322 includes a socket 332 for holding the flange 320 and a hose connector 334. The hose 310 is connected to the hose connector 334 to provide suction force from the vacuum pump 214 to the flange 320.

As shown in FIGS. 4A-4B, the flange 320 has a cylindrical coupling 340 that is connected to the socket 332 on the valve housing 322. The coupling 340 is connected to a neck member 342 that is connected to a cup member 344. The opposite side of the flange 320 from the coupling 340 has a substantially circular rim interface 346 that contacts the skin of the user.

The substantially circular rim interface 346 includes a contact layer 348 having a microtexture that assists in seal performance and comfort. In this example, the neck member 342 and the cup member 344 are fabricated from silicone. As will be explained below, the contact layer 348 is constructed of material to facilitate conform in the interface with the skin of the user.

The flange interface with the skin of user may be improved by increasing performance and comfort through the contact layer 348 described herein. Performance is achieved by enhancing seal performance of the flange 320 on the breast of the user. The interface between the flange 320 and the skin must be able to seal to a range of different breast profiles. In this example, the interface has improved geometry based on microtextures and materials that assist with sealing. In addition, seal and comfort is improved by the selection of tactile surface finishes and/or material selection that improve compliance and tactile comfort. In this example, the rim interface 346 may include a silicone interface that may be flocked or comprise particular surface microtextures to improve the surface feel or reduce friction, thereby improving comfort. In addition, a surface feature such as adhesive sections that attach to a skin surface may be added to improve seal performance by better engagement with the breast.

Seal and comfort may also be improved by material selection. For example, foam interfaces are known to improve seal and comfort due to improved compliance. Foam also has an increased spring rate over materials such as silicone or plastic flange seals. Foam may also improve seal and comfort by evenly distributing load on the breast during use. Alternatively, textile technology enabling complete 3D structures to be produced may be used. Thus, textile seals with complex geometries can be constructed to improve seal and comfort of the breast interfaces. Textiles are known to be more stretchable than silicone, while being able to maintain significantly thin cross sections in use.

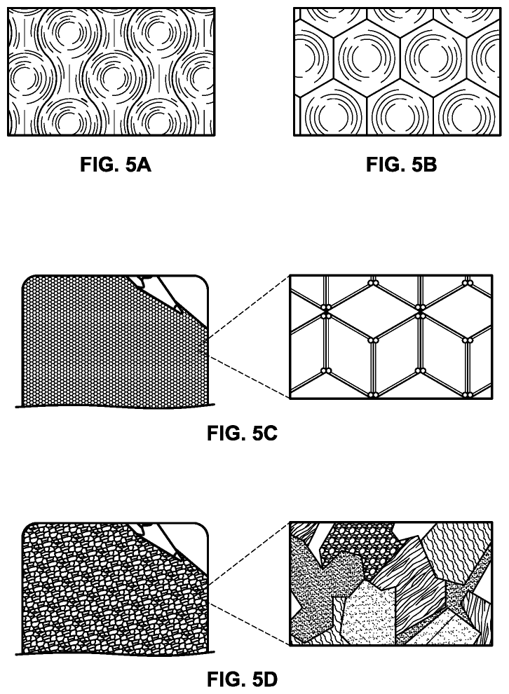

FIGS. 5A-5H are actual sized and microscopic images of different microtextures that may be applied to the surface of the contact layer 348 to create better contact with the skin of a user. Specifically FIG. 5A shows a high magnification image of a Reichle molecule texture. FIG. 5B shows a high magnification image of another microtexture. FIG. 5C shows an actual sized image and a high magnification image of a polished complex surface that has very high polish. FIG. 5D shows an actual sized image and a high magnification image of a polished complex surface that has very high polish. FIG. 5E shows an actual sized image and a high magnification image of a polished complex surface that has very high polish. FIG. 5F shows an actual sized image and a high magnification image of a surface with microtextured depth modulation. FIG. 5G shows an actual sized image and a high magnification image of a surface having frit/vignette microtextured depth modulation. FIG. 5H shows an actual sized image and a high magnification image of a surface having a microtexture blend of carbon fiber to leather.

Each of the microtextures in FIGS. 5A-5H create an artificial textile-like hand feel in the silicone of the flange 320. Another alternative is a process known as Parylene Coating and involves a series of vapor-deposited coatings onto the flange surfaces. The coating creates a smooth, soft touch, velvet-esque texture that provides greater comfort for the user.

The flange 320 in FIGS. 4A-4C may be custom manufactured for a particular user to enhance comfort and compatibility. For example, a scan may be taken of the breast area of a user and the image data may be used to construct a customized interface surface and shape for the flange 320. The user may be provided with a scanning application on the mobile device 120 to scan the upper front of their body from left to right and back again. A sizing application executing on the mobile device 120 or a remote server could then process the data and calculate the cup size of the breast. With the different breast pump systems and flange sizes, a recommendation could be made on the optimally sized components for interface with the individual user. Alternatively, a customized flange could be provided based on the body data from the scan.

In one example, the scanning application may be downloadable from a manufacturer or third party server to a smartphone or tablet with an integrated camera. When launched, the application may provide visual and/or audio instructions. As instructed, the user may stand in front of a mirror, and press the camera button on a user interface. An activated process may then take a series of pictures of the area around the breast, and then, within a matter of seconds for example, recommend a flange size for the user (based on analysis of the pictures). The scanning application allows a user, anywhere in the world, to quickly and conveniently find a flange suitable for their needs. Moreover, the user may repeat the scan process if desired, unhurriedly and to their satisfaction, increasing the confidence and sense of responsibility of the user.

As described further below, the present technology allows a user to capture an image or series of images of the breast area. Instructions provided by an application stored on a computer-readable medium, such as when executed by a processor, detect various landmarks within the images, measure and scale the distance between such landmarks, compare these distances to a data record, and recommend an appropriate flange. Thus, an automated device of a user may permit accurate flange selection, such as in the home, to permit users to determine sizing without trained personnel to assist them.

The image sensor may be one or more cameras (e.g., a CCD charge-coupled device or active pixel sensors) that are integrated into a computing device, such as those provided in a smartphone such as the mobile device 120 in FIG. 1 or in a laptop. Alternatively, where the computing device is a desktop computer, the computer may include a sensor interface for coupling with an external camera, such as a webcam. Other exemplary sensors that could be used to assist in the methods described herein that may either be integral with or external to the computing device include stereoscopic cameras, for capturing three-dimensional images, or a light detector capable of detecting reflected light from a laser or strobing/structured light source.

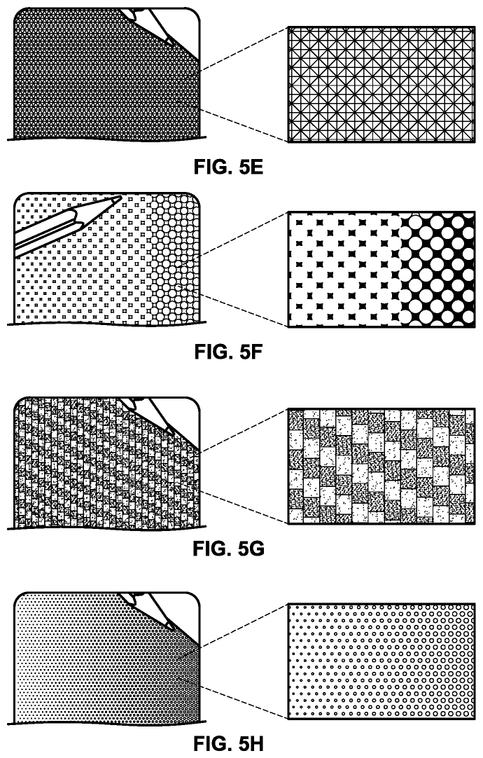

As illustrated in the flow diagram of FIG. 6, an example scanning application allows measurement of breast area features using two-dimensional or three-dimensional images and recommendation or selection of an appropriate flange, such as from a group of standard sizes, based on the resultant measurements. The method may generally be characterized as including three or four different phases: a pre-capture phase, a capture phase, a post-capture image processing phase, and a comparison and output phase.

In this example, the scanning application may cause a visual display to be output that includes a reference feature on the display of a computing device such as the mobile device 120. The user may position the feature adjacent to their chest, such as by movement of the camera. The processor may then capture and store one or more images of the features in association with the reference feature when certain conditions, such as alignment conditions are satisfied. This may be done with the assistance of a mirror. The mirror reflects the displayed reference feature and the chest of the user to the camera. The application then controls the processor to identify certain features within the images and measure distances therebetween. By image analysis processing a scaling factor may then be used to convert the feature measurements, which may be pixel counts, to standard flange measurement values based on the reference feature. Such values may be, for example, standardized unit of measure, such as a meter or an inch, and values expressed in such units suitable for flange sizing.

Additional correction factors may be applied to the measurements. The feature measurements may be compared to data records that include measurement ranges corresponding to different flange sizes for particular user features. The recommended size may then be chosen and be output to the user based on the comparison(s) as a recommendation. Such a process may be conveniently performed within the comfort of the user's own home, if the user so chooses. The application may perform this method within seconds. In one example, the application performs this method in real time.

In the pre-capture phase, the scanning application, among other things, assists the user in establishing the proper conditions for capturing one or more images for sizing processing. Some of these conditions include proper lighting and camera orientation and minimal motion blur caused by an unsteady hand holding the computing device, for example.

When the user launches the scanning application, the application may prompt the user via the display interface of the computing device to provide user specific information, such as age, gender, weight, and height. However, the scanning application may prompt to the user to input this information at any time, such as after the features of the user are measured. The scanning application may also present a tutorial, which may be presented audibly and/or visually, as provided by the scanning application to aid the user in understanding their role during the process. Also, in the pre-capture phase, the scanning application may extrapolate the user specific information based on information already gathered by/from the user, such as after receiving captured images of the user, and based on machine learning techniques or through artificial intelligence.

When the user is prepared to proceed, which may be indicated by a user input or response to a prompt, the application activates an image sensor (600). The image sensor is preferably the forward facing camera of the mobile device 120, for example, which is located on the same side of the mobile device as the display. The camera is generally configured to capture two-dimensional images. Mobile device cameras that capture two-dimensional images are ubiquitous. The present technology takes advantage of this ubiquity to avoid burdening the user with the need to obtain specialized equipment.

Around the same time the image sensor/camera is activated, the scanning application presents a capture interface on the display (602). The capture interface may include a camera live action preview, a reference feature, a targeting box, and one or more status indicators or any combination thereof. The reference feature is a feature that is predetermined and provides a frame of reference to allow the scanning application to scale captured images. The reference feature may preferably be a feature other than an anatomical feature of the user. Thus, during the image processing phase, the reference feature assists the scanning application in determining when certain alignment conditions are satisfied, such as during the pre-capture phase. The reference feature may be a quick response (QR) code or other known exemplar or marker, which can provide the scanning application certain information, such as scaling information, orientation, and/or any other desired information which can optionally be determined from the structure of the QR code. The QR code may have a square or rectangular shape. When displayed on the display, the reference feature has predetermined dimensions, such as in units of millimeters or centimeters, the values of which may be coded into the scanning application. The actual dimensions of the reference feature may vary between various computing devices. In some versions, the application may be configured to be a computing device model specific application in which the dimensions of the reference feature, when displayed on the particular model, are already known. However, in other embodiments, the scanning application may obtain certain information from the computing device, such as display size and/or zoom characteristics that allow determining the real world/actual dimensions of reference feature as displayed on the display via scaling. Regardless, the actual dimensions of reference feature as displayed on the display interfaces of such computing devices are generally known prior to post-capture image processing.

In this example, when the user holds the display parallel to the chest features to be measured and presents the user display to a mirror or other reflective surface, the reference feature is prominently displayed and overlies the real-time images seen by the camera/sensor and as reflected by the mirror. This reference feature may be fixed near the top of display. The reference feature is prominently displayed in this manner at least partially so that sensor can clearly see the reference feature so that the scanning application can easily identify the reference feature. In addition, the reference feature may overlie the live view of the chest of the user, which helps avoid user confusion.

Other features or information can be displayed on the display. For instance, the scanning application may establish parameters that must be satisfied regarding lighting conditions. If lighting conditions are unsatisfactory, the scanning application may display a warning on the display or output an audible warning to the user and instruct the user on steps that can be taken that can help rectify the unsatisfactory lighting conditions.

The user may position themselves or their chest and the mobile device 120 in front of a mirror such that the chest of the user and the display of the mobile device 120 are reflected back to the image sensor. The user may also be instructed by visual instruction, by audible instructions via a speaker of the mobile device 120, or be instructed ahead of time by the tutorial, to position the display in a plane of the chest features to be measured. For example, the user may be instructed to position the display in a plane aligned with certain chest features to be measured. As the images ultimately captured are two-dimensional, planar alignment (604) helps ensure that the scale of the reference feature is equally applicable to the feature measurements. In this regard, the distance between the mirror and the features of chest of the user and the display will be approximately the same.

When the user is positioned in front of the mirror, the display, which includes the reference feature, is roughly placed in planar alignment with the chest features to be measured, the scanning application checks for certain conditions to help ensure sufficient alignment when the reference feature is read (606). One exemplary condition that may be established by the application, is that the entirety of the reference feature must be detected within the targeting box in order to proceed (608). If the scanning application detects that reference feature is not entirely positioned within the targeting box, the user may be instructed to move their chest along with the display to maintain planarity until the reference feature, as displayed in the live action preview, is located within targeting box. This helps optimized alignment of the features and display with respect to the mirror for image capture.

When the scanning application detects the entirety of reference feature within the targeting box (608), the application may read an inertial measurement unit (IMU) of the mobile device 120 for detection of device tilt angle (610). The IMU may include an accelerometer or gyroscope, for example. Thus, the scanning application may evaluate device tilt such as by comparison against one or more thresholds to ensure it is in a suitable range. For example, if it is determined that mobile device 120, and consequently the display and features of the chest of the user, is tilted in any direction within about +/−5 degrees (612), the process may proceed to the capture phase. In other embodiments, the tilt angle for continuing may be within about +/−10 degrees, +/−7 degrees, +/−3 degrees, or +/−1 degree. If excessive tilt is detected a warning message may be displayed or sounded to correct the undesired tilt. This is particularly useful for assisting the user to help prohibit or reduce excessive tilt, particularly in the anterior-posterior direction, which if not corrected, could pose as a source of measuring error as the captive reference image will not have a proper aspect ratio.

The capture phase includes initiating capture of the image (614). The capture phase preferably occurs automatically once the alignment parameters and any other conditions precedent are satisfied. However, in some embodiments, the user may initiate the capture in response to a prompt to do so. When image capture is initiated, the scanning application captures a number n of images via the image sensor (616), which is preferably more than one image. For example, about 5 to 20 images, 10 to 20 images, or 10 to 15 images may be captured, etc. The quantity of images captured may be time based. In other words, the number of images that are captured may be based on the number of images of a predetermined resolution that can be captured by the image sensor during a predetermined time interval. For example, if the number of images the image sensor can capture at the predetermined resolution in 1 second is 40 images and the predetermined time interval for capture is 1 second, the image sensor will capture 40 images for processing. The quantity of images may be user-defined, determined by a server based on artificial intelligence or machine learning of environmental conditions detected, or based on an intended accuracy target. For example, if high accuracy is required then more captured images may be required. Although, it is preferable to capture multiple images for processing, one image is contemplated and may be successful for use in obtaining accurate measurements. However, more than one image allows average measurements to be obtained. This may reduce error/inconsistencies and increase accuracy. The images may be placed in the storage of the mobile device 120.

The scanning application then proceeds to the post-capture image processing phase. Once the images are captured, the images are processed to detect or identify features/landmarks and measure distances therebetween. The resultant measurements may be used to recommend an appropriate flange size. This processing may alternatively be performed by an application on a server receiving the transmitted captured images, or on the computing device. Processing may also be undertaken by a combination of the computing device and server. The scanning application retrieves one or more captured images from stored data (618). The image is then extracted by to identify each pixel comprising the two-dimensional captured image. The scanning application then detects certain pre-designated features within the pixel formation of the retrieved image (620).

Detection may be performed by using edge detection, such as Canny, Prewitt, Sobel, or Robert's edge detection, for example. These edge detection techniques/algorithms help identify the location of certain features within the pixel formation, which correspond to the actual chest features as presented for image capture. The scanning application may then mark, tag or store the particular pixel location(s) of each of these features. Alternatively, or if such detection by the scanning application is unsuccessful, the pre-designated features may be manually detected and marked, tagged or stored by a human operator with viewing access to the captured images.

Once the pixel coordinates for these features are identified, the scanning application measures the pixel distance between certain of the identified features (622). For example, the distance may generally be determined by the number of pixels for each feature and may include scaling. Once the pixel measurements of the pre-designated features are obtained, an anthropometric correction factor(s) may be applied to the measurements (624). It should be understood that this correction factor can be applied before or after applying a scaling factor, as described below. The anthropometric correction factor can correct for errors that may occur in the automated process, which may be observed to occur consistently from user to user. In other words, without the correction factor, the automated process, alone, may result in consistent results from user to user, but results that may lead to a certain amount of mis-sized flanges. The correction factor, which may be empirically extracted from population testing, shifts the results closer to a true measurement helping to reduce or eliminate mis-sizing. This correction factor can be refined or improved in accuracy over time as measurement and sizing data for each user is communicated from respective computing devices to a server where such data may be further processed to improve the correction factor. The anthropometric correction factor may also vary between the forms of flanges.

In order to apply the feature measurements to user flange sizing, whether corrected or uncorrected by the anthropometric correction factor, the measurements may be scaled from pixel units to other values that accurately reflect the distances between the features of the user as presented for image capture. The reference feature may be used to obtain a scaling value or values. Thus, the scanning application similarly determines dimensions of the reference feature (626), which can include pixel width and/or pixel height (x and y) measurements (e.g., pixel counts) of the entire reference feature. More detailed measurements of the pixel dimensions of the many squares/dots that comprise a QR code reference feature, and/or pixel area occupied by the reference feature and its constituent parts may also be determined. Thus, each square or dot of the QR code reference feature may be measured in pixel units to determine a scaling factor based on the pixel measurement of each dot and then averaged among all the squares or dots that are measured, which can increase accuracy of the scaling factor as compared to a single measurement of the full size of the QR code reference feature. However, it should be understood that whatever measurements are taken of the reference feature, the measurements may be utilized to scale a pixel measurement of the reference feature to a corresponding known dimension of the reference feature.

Once the measurements of the reference feature are taken, the scaling factor is calculated by the application (628). The pixel measurements of reference feature are related to the known corresponding dimensions of the reference feature, e.g. the reference feature as displayed for image capture, to obtain a conversion or scaling factor. Such a scaling factor may be in the form of length/pixel or area/square pixel. In other words, the known dimension(s) may be divided by the corresponding pixel measurement(s) (e.g., count(s)).

The scaling factor is then applied to the feature measurements (pixel counts) to convert the measurements from pixel units to other units to reflect distances between the actual features suitable for flange sizing (630). This may typically involve multiplying the scaling factor by the pixel counts of the distance(s) for features pertinent for flange sizing.

The application then determines whether the end of the image set has been reached (632). If there are further images, the measurement steps and calculation steps for both the features and reference feature are repeated for each captured image until each image in the set has feature measurements that are scaled and/or corrected.

The corrected and scaled measurements for the set of images may then optionally be averaged or otherwise combined to obtain final measurements of the chest features (634). Such measurements may reflect distances between the features of the user. The application then proceeds to the comparison and output phase. The scanning application then may display, forward and compare the averaged results (636). Thus, the results from the post-capture image processing phase may be directly output (displayed) to a person of interest or compared to data record(s) to obtain an automatic recommendation for a user flange size.

Once all of the measurements are determined, the results (e.g., averages) may be displayed on the mobile device 120. In one embodiment, this may end the automated process. The user can record the measurements for further use by the user.

Alternatively, the final measurements may be forwarded either automatically or at the command of the user to a server for conducting further processing and analysis to determine a suitable flange for the user. In a further embodiment, the final feature measurements that reflect the distances between the actual features are compared to flange size data such as in a data record. The data record may be part of the scanning application. This data record can include, for example, a lookup table, which may include flange sizes corresponding to a range of feature distances/values. Multiple tables may be included in the data record, many of which may correspond to a particular form of a flange and/or a particular model of a flange offered by the manufacturer.

The scanning application compares the measurements to determine an appropriate size or “best fit,” such as by identifying one or more ranges within which the measurements fall and then selecting the flange size, such as from a group of standard flanges (e.g., small, medium, or large, etc.), associated with that identified range(s) (638). The scanning application may then recommend the identified flange size in the form of a display presented on the display of the mobile device. The scanning application or a server may also automatically forward the recommendation via email, text message or instant messenger for the user. Alternatively, the measurements, if of sufficient detail, may be used to fabricate a custom sized flange specific to the individual user.

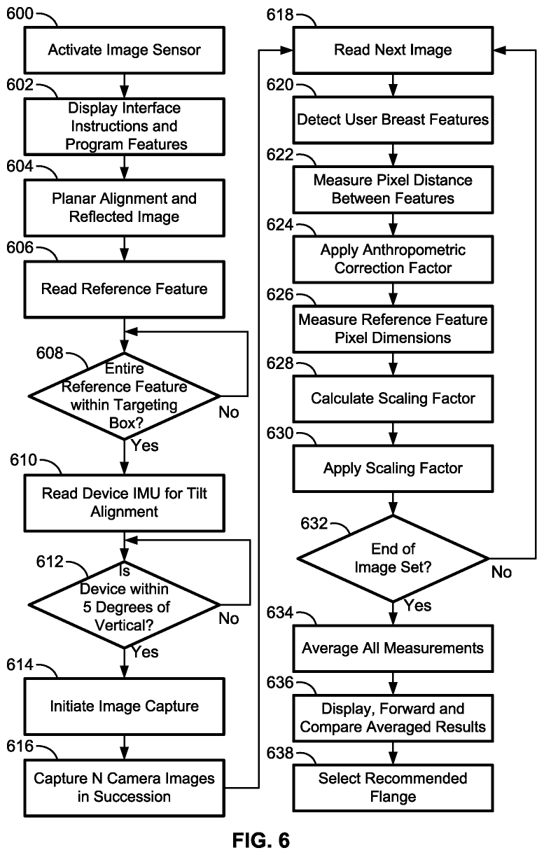

The system 100 allows collection of data for determination of individualized settings of the pump device 112. FIG. 7 is a flow diagram of the process of collection of data for the purposes of determining individual settings for the pump device 112. A user first uses the breast pump system 110 and pumps milk at initial settings and for an initial duration (700). The user will finish using the pump device 112 (702). During the operation, the controller 210 collects operational data such as pressure data from the pressure sensor 250 and stores the data internally. Alternatively, such data may be transmitted via the communication module 218 to an external device such as the mobile device 120 in FIG. 1. The process memorizes the settings used during the pump session and provides parameters on pressure, duration, and other statistics (704). The parameters are stored in a database (706). In this example, the database may be accessible by data servers such as the data server 154 in FIG. 1.

The application server may execute an optimization algorithm to retrieve the stored parameters and adjust the settings (708). For example, the optimal pressure generated by the pump for a user may be communicated. The adjusted settings are then communicated back to the pump device 112 and used for the next use of the pump device 112.

One example of a personalized setting is an “autoset” mode. The autoset mode will first learn the user preference after an initial manual set up of the pump device 112 by the user. As shown in FIG. 7, the autoset mode will provide a customized mode that utilizes the user preferences obtained during the set up for settings upon subsequent use of the pump device 112. For example, the user may manually set the pump device 112 to an initial prime mode, which massages the breast to simulate breast feeding. In the prime mode, the negative pressure in the breast interface surrounding the breast fluctuates. This prime mode is intended to initiate milk production via the “let-down” reflex, which occurs in response to stimulus equivalent to a baby's suckling action.

Once milk starts to produce, the user may switch to a pump mode, which is a more constant negative pressure that continues to extract milk from the breast. The autoset mode, via the application 130 running on the mobile device 120 in conjunction with the controller 210 on the pump device 112, will learn the user inputs e.g. the duration of the prime mode, when the pump mode was activated, and how much milk is produced. The learned user inputs will be stored on either the pump device 112 or an external device such as the mobile device 120 and the preferred settings may be generated from this data.

The application 130 may control the vacuum pump 214 via preprogrammed algorithms to improve the pumping experience. The vacuum pump 214 may be controlled to generate a desirable negative pressure in the flange 320 to enhance pumping efficacy resulting in higher milk production and also reduce discomfort. The settings may be auto-selected via storage of previous inputs into the pump device 112 or the application 130 by the user. In addition, the pump device 112 and/or the application 130 may track efficacy via the pressure sensor 250 and learn optimal settings for future use and adapt the settings accordingly to control the output of the vacuum pump 214.

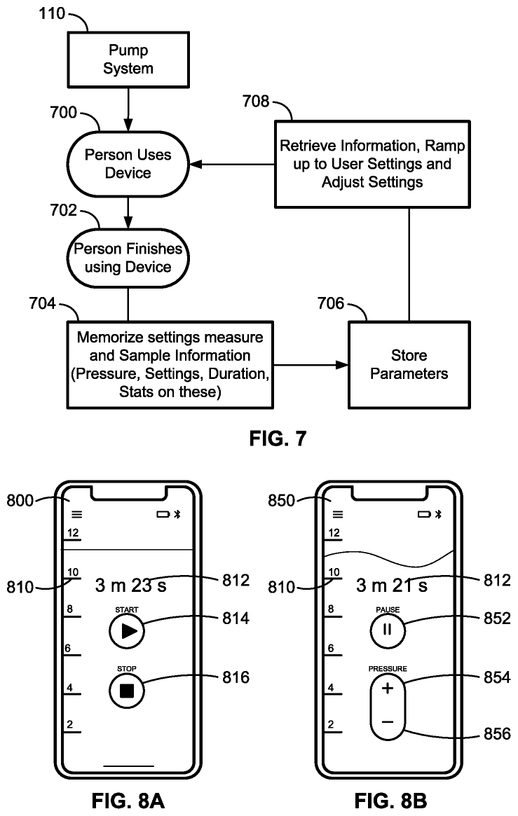

Examples of control interfaces that may be generated by the application 130 on the display of the mobile device are shown in FIGS. 8A-8B. FIGS. 8A-8B are screen images of user interfaces on the mobile device 120 associated with a user of the pump system 110 in FIG. 1. As shown in FIG. 8A, a first control interface 800 is generated that includes a milk volume scale 810, a time field 812, a start button 814 and a stop button 816. The data for generating the interface 800 is provided by the pressure sensor 250 in the pump device 112 and other relevant background data.

The interface 800 allows a user to control the vacuum pump 214 in FIG. 2 via the mobile device 120. For example, a user may start the vacuum pump 214 by touching the start button 814, which causes the vacuum pump 214 to activate the suction to the flange 320 and begin the milking process. The user may stop the vacuum pump 214 by touching the stop button 816. The time field 812 shows duration of the pumping based on the start and stop time of the pump 214.

FIG. 8B shows a second control interface 850 that includes the milk volume scale 810 and time field 812 similar to the interface 800 in FIG. 8A. The interface 850 includes a pause button 852, an increase pressure arrow 854, and a decrease pressure arrow 856. The arrows 854 and 856 allow a user to increase or decrease the pressure generated by the vacuum pump 214 via control of the suction generated by the vacuum pump 214. A background line 860 in the interface 850 changes depending on the actual volume of milk pumped. During pumping operation, the average height of the line 860 changes in accordance with the volume scale 810 and the line 860 fluctuates in a wave pattern. As shown in FIG. 8A, when the pump is not activated, the line 860 is flat and reflects of the actual milk volume expressed. Once the pump is started, the application 130 may switch to the interface 850 in FIG. 8B to allow the user to control the pressure of the pump during the session via the up and down arrows 854 and 856. A user may also pause the pumping operation by the pause button 852. Using the accelerometer in the mobile phone the application 130 can make the level “tilt.”

All usage information may be tracked and logged e.g. how often the pump device 112 was used, when, where and how much milk was produced in accordance with the application 130. This information may be provided back to the user to track progress during milking operation as shown by the example interfaces in FIGS. 8A-8B. Additional information may be processed by the application 130 to also determine optimal usage times and settings for the user. The information may also be provided to an external server that may provide applications accessible by clinicians/lactation consultants, so they may tailor advice for the user.

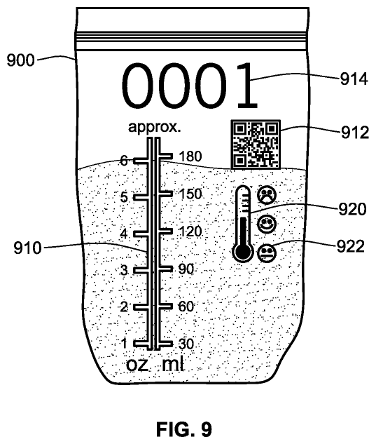

FIG. 9 shows a perspective view of a bag 900 that may be tracked for milk production. The bag 900 may be substituted for the bottle container 326 in the above description. The breast milk containers such as the bag 900 may enable tracking of stored milk via unique QR codes or other identifiers that may be scanned by a smart phone and tracked via a database in the application 130 or on an application on a remote server. The bag 900 in this example is constructed of clear plastic to show the milk contained. The bag 900 has a volume scale 910 that shows the approximate volume of milk contained in the bag 900 (in the illustrated example, the volume is approximately 180 milliliters or 6 oz.). The bag 900 includes a unique identification tag 912, a QR code in this example, that encodes a unique identification number associated with the bag 900. The identification tag may alternatively be a bar code or other machine-scannable insignia. A printed identification number 914 may be provided to allow a user to visually identify the bag 900.

The bag 900 may comprise a visual temperature indicator 920, which indicates when the milk is too hot to drink. The indicator 920 may include icons 922 that visually provide information relating to the proper temperature for the stored milk.

The mobile device 120 may be used to capture the code from the identification tag 912 and mate it with relevant data obtained from the pump device 112. For example, a QR code may be scanned by the camera of a mobile device and stored along with data such as the amount of the milk in the bag 900 as well as the time the milk was pumped. In this manner, a server based application may track the bags for purposes of storage of milk for later use in terms of the optimal time to feed the milk to a baby. Each user may be provided an inventory interface generated by the application 130 showing the number of available bags of milk based on the stored identification and associated data. The user may be informed of data such as when the bag was filed, how much milk was produced and when it is expected to expire via an interface generated by the application 130. The application 130 may also provide a systematic scheduling of when to feed a baby the milk in a specific bag based on the optimization of the available bags from the data related to each bag.

The system 100 may also provide fleet management of pump consumables via the supplier system 160 in FIG. 1. The application 130 may have a portal to online resupply of consumables such as that managed by the supplier system 160 in FIG. 1. The application 130 may track usage and provide reminders or an automated ordering function to replace consumables as required.

The estimates of remaining usage time of the motor of the vacuum pump 214 may be further utilized by the various entities in the system 100. In one example, the remaining capacity and/or usage time estimate of different system components of the pump device 112 may be displayed on the display of the pump device 112. For example, LEDs or icons may be used to indicate the current value of the remaining capacity estimate (e.g. 100%, 75%, 50%, 25%). This display may occur in response to activation of a separate button on the control panel 304 of the pump device 112 or on the interface generated by the application 130 on the mobile device 120.

This could occur on the instruction of a server such as a server of the supplier system 160 via a “push notification” to the application, or on the initiative of the application itself based on the need to replace one of the components. This is one example of the kind of rule-based fleet management made possible by estimating pump component or flange life.

Optionally, such as in case where the application 130 or an internal routine on the pump device 112 estimates the remaining lifetime of the components, the pump device 112 may communicate a message, which may be based on the estimate, such as by a comparison with a threshold (e.g., if the estimate is at or below a threshold), to an external computing device of the supplier system 160 such as to provide a notification message of a replacement component such as pump motor or a new flange, or additional storage containers. Such a message may be a request for a new batch of bags or bottles, such as for arranging a purchase or replacement order via an ordering or fulfillment system implemented with any of the devices managed by the system 100. Such a message may also be generated by any of the devices of the system 100 that receives either the estimate or the measurements and parameters necessary for determining the estimate. In such a case, the message may be further transmitted to other systems, such as a purchasing, ordering or fulfillment system or server(s) that may be configured to communicate with a device of the system 100 for arranging and/or completing such orders. Still further, in some versions, the pump device 112 may make a change in a control parameter of the pump device 112 based on the estimate or a comparison of the estimate and one or more thresholds. For example, one or more parameters for control of the pressure generated by the pump device 112 may be adjusted based on the comparison. Such adjustments may include, for example, reducing the necessary pressure during certain pumping sessions that may increase the life of the different components.

The system 100 in FIG. 1 may be configured to transmit electronic messages and other media to user computing devices such as the mobile devices 120. The messages or media may be managed by a server such as the clinical server 150. The messages may be in various modes of engagement such as emails, SMS messages, automated voice messages, or notifications. Some such messages may prompt the user for a response via the same mode. For example, an SMS message may prompt the user to acknowledge that they have read and understood the message. Such responses are transmitted from the mobile device 120 to the data server 154, where they are stored as “engagement data.” If no response was received when prompted for, the engagement data may represent that fact.

In some implementations, the data server 154 is configured to communicate with the HCP server 140 to trigger notifications or action recommendations to an agent of the HCP such as a nurse, or to support reporting of various kinds. Examples of recommended actions include phone calls and personal visits to the user by a nurse or technician. Such actions are also used to implement certain forms of engagement as part of the user's natal care program. Details of actions carried out are stored by the data server 154 as part of the engagement data.

The application 130 may provide a portal to have video links to lactation consultants and doctors through the clinical server 150. For example, the application 130 may provide the user with links to educational media on pumping or access to social groups. Further, the application may provide other media for assistance to the user. For example, messages may be sent to provide instructions for user of the breast pump system 110. Links to videos for instructions such as for using the breast pump system 110, or for detecting and fixing leaks may be provided based on data from the operation of the breast pump system 110. The application 130 may also provide positive reinforcement of usage e.g. rewards for using the breast pump system 110. The app may provide the user with pre-set or customized scheduling of pumping e.g. calendar reminders and assistance to prevent mastitis.

In some implementations, the data server 154 may be configured to transmit control commands to the pump device 112. A control command may be an instruction to adjust a setting of the pump device 112 in response to received data to optimize pumping of milk in accordance with changed environmental or health data of the user.

The data server 154 may host an optimization application that analyzes data from the breast pump system 110, the mobile device 120, the sensors 116, the HCP server 140, and the EMR server 144, to generate a prediction about the settings for the breast pump system 110 to tailor settings for the user. The optimization application selects an action intended to improve the operation of the breast pump system 110 specific to the user.

The data server 154 analyses the data available to it in order to generate a prediction about the settings for a specific user. The data available to the data server 154 and may comprise one or more of the following: profile data, behavioral data, physiological data, summary data, EMR data, and HCP data.

The profile data may include demographic data such as user age, marital status, weight, occupation, address, education level, and nationality, and the primary care physician who is monitoring natal care. The profile data may also include details including type and model of the components of the breast pump system 110, the initial settings of the breast pump system 110, and type, model, and size of the flange unit 114 to be used. In one implementation of the user enters the profile data to the application 130, and the mobile device 120 transmits the profile data to the data server 154. In another implementation, an operator of the HCP server 140 enters the profile data manually to the HCP server 140 via an HCP server process, and the HCP server process transmits the profile data to the data server 154.

The usage data could also be provided to insurers such as operators of the payor server 152 to track device usage. Data may also be collected to provide commercial insights.

The settings of the breast pump system 110 are able to be adjusted remotely via a control command to the breast pump system 110 to change its settings in accordance with the selected adjustment. If the settings of the breast pump system 110 are not able to be adjusted remotely, the data server 154 may send a message to the user via the mobile device 120 to prompt the user to adjust the settings of the breast pump system 110. In another such implementation, the data server 154 sends a message to the HCP server 140 to prompt a technician or health care professional to be dispatched to the user to adjust the settings of the breast pump system 110.

The flow diagrams in FIGS. 6 and 7 are representative of example machine readable instructions for determining settings for the breast pump system and selection of interface components based on a scan. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowcharts illustrated in FIGS. 6 and 7, persons of ordinary skill in the art will readily appreciate that many other methods of implementing the example machine readable instructions may alternatively be used. For example, the order of execution of the blocks may be changed, and/or some of the blocks described may be changed, eliminated, or combined.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

LABEL LIST

| | |
|---|---|
| support system | 100 |
| breast pump system | 110 |
| pump device | 112 |
| flange unit | 114 |
| sensors | 116 |
| consumables | 118 |
| mobile device | 120 |
| application | 130 |
| health care provider server | 140 |
| mobile device | 142 |
| EMR server | 144 |
| clinical server | 150 |
| payor server | 152 |
| data server | 154 |
| database | 156 |
| machine learning applications | 158 |
| supplier system | 160 |
| main controller unit | 210 |
| battery | 212 |
| vacuum pump | 214 |
| pressure sensor unit | 216 |
| communication module | 218 |
| wireless charger | 220 |
| mains power unit | 222 |
| milk container | 230 |
| pressure sensor | 250 |
| breast interface port | 252 |
| housing | 300 |
| display | 302 |
| control panel | 304 |
| hose | 310 |
| flange | 320 |
| valve housing | 322 |
| coupling | 324 |
| bottle | 326 |
| hemispheric cover | 330 |
| socket | 332 |
| hose connector | 334 |
| coupling | 340 |
| neck member | 342 |
| cup member | 344 |
| rim interface | 346 |
| contact layer | 348 |
| user | 400 |
| step | 600 |
| step | 602 |
| step | 604 |
| step | 608 |
| step | 610 |
| step | 612 |
| step | 614 |
| step | 616 |
| step | 618 |
| step | 620 |
| step | 622 |
| step | 624 |
| step | 626 |
| step | 628 |
| step | 630 |
| step | 634 |
| step | 636 |
| step | 638 |
| step | 700 |
| step | 704 |
| step | 706 |
| step | 708 |
| interface | 800 |
| volume scale | 810 |
| time field | 812 |
| button | 814 |
| button | 816 |
| interface | 850 |
| pause button | 852 |
| arrow | 854 |
| arrow | 856 |
| background line | 860 |
| bag | 900 |
| volume scale | 910 |
| identification tag | 912 |
| identification number | 914 |
| indicator | 920 |
| icons | 922 |

1. A breast pump device comprising:
a vacuum pump to provide suction force to a flange, the flange configured to attach to a chest area of a user around a breast of the user, the vacuum pump having different settings to control the suction force during a pumping operation; and
a controller is configured to control the pump, the controller operable to provide different modes of pumping operation, the modes comprising an initial prime mode to provide fluctuating negative pressure within the flange to simulate feeding, and a pump mode providing a more constant negative pressure.

2. The breast pump device of claim 1, further comprising a transceiver in communication with an external device, the transceiver configured to:
transmit data associated with the pumping operation; and
receive control commands for the controller.

3. The breast pump device of claim 1, wherein the external device is operative to learn optimal settings for the user based on transmitted data specific to the user.

4. The breast pump device of claim 3, wherein the transmitted data include at least one of: the duration of the prime mode, how much milk is produced, and the pressure within the flange during the pumping operation.

5. The breast pump device of claim 1, wherein the optimal settings are provided in the control commands by the external device.

6. The breast pump device of claim 1, wherein the optimal setting is a time for pumping operation.

7. The breast pump device of claim 1, wherein the external device is a mobile device associated with the user.

8. The breast pump device of claim 7, wherein the mobile device is in communication with an application server.

9. The breast pump device of claim 1, wherein the external device is a networked server.

10. The breast pump device of claim 1, further comprising a pressure sensor coupled to the flange, the pressure sensor configured to provide pressure data associated with the pumping operation.

11. A breast pump device comprising:
a vacuum pump to provide suction force to a flange, the flange configured to attach to a chest area of a user around a breast of the user, the vacuum pump having different settings to control pressure of the suction force;
a pressure sensor coupled to the flange, the pressure sensor configured to generate pressure data representative of air pressure in the flange;
a controller configured to control the pump; and
a transceiver in communication with an external device, the transceiver configured to transmit pressure data received by the controller from the pressure sensor.

12. The breast pump device of claim 11, wherein the pressure sensor is a gauge type transducer.

13. The breast pump device of claim 11, wherein the controller is further configured to determine the duration of a pumping session, and send the duration data to the transceiver.

14. The breast pump device of claim 13, wherein the external device is operable to determine an estimate of milk production based on the received pressure and duration data.

15. The breast pump device of claim 11, wherein the external device is operable to determine the pressure required to be applied by the pump to draw milk.

16. The breast pump device of claim 11, wherein the pressure data is used to determine an air leak based on a decrease in sensed negative pressure.

17-21. (canceled)

22. A support system for operation of a breast pump device, the breast pump device including a vacuum pump to provide suction force to a flange, the flange configured to attach to chest area of a user around a breast of the user, a controller configured to control the pump according to different modes of pumping operation, the modes comprising an initial prime mode to provide fluctuating negative pressure within the flange to simulate feeding, and a pump mode providing a more constant negative pressure; and a transceiver, the system comprising:
a network interface for receiving data associated with the pumping operation from the transceiver;
a database coupled to the network interface to store the data received from the transceiver, the database including data associated with the user; and
a processor operable to collect the data received from the transceiver, process the data, and provide the processed data to the database.

23. (canceled)

24. The support system of claim 22, wherein the processor is operable to determine initial settings for the pump device based on analysis of data from a plurality of breast pump devices including the breast pump device.

25. The support system of claim 23, wherein the processor is operable to determine optimal scheduling for pumping based on analysis of data from the plurality of breast pump devices.

26. The support system of claim 22, wherein the processor is operable to determine a time for replacement of one of the group of components of the pump device consisting of: the vacuum pump, and the flange.

27-38. (canceled)
